(19) United States
(12) Patent Application Publication
(10) Pub. No.: US 2012/0070830 A1
(43) Pub. Date: Mar. 22, 2012

(54) STABILIZATION OF OZONE-LABILE FLUORESCENT DYES BY THIOUREA

(75) Inventors: Mark W. Eshoo, Solana Beach, CA (US); John Picuri, Carlsbad, CA (US)

(73) Assignee: lbis Biosciences, Inc., Carlsbad, CA (US)

(21) Appl. No.: 13/233,913

(22) Filed: Sep. 15, 2011

Related U.S. Application Data
(60) Provisional application No. 61/383,603, filed on Sep. 16, 2010.

(51) Int. Cl. C12Q 1/68; C07D 403/02

(52) U.S. Cl. 435/6.11; 435/6.12; 548/401

(57) ABSTRACT

The present invention provides compositions and methods for stabilization of fluorescent dyes. In particular, the present invention provides buffer systems comprising thiourea to protect against degradation of ozone-labile fluorescent dyes.

### CROSS-REFERENCE TO RELATED APPLICATIONS

The present Application claims priority to PCT Application No. ______ filed Sep. 15, 2011 (filed concurrently herewith) and U.S. Provisional Application Ser. No. 61/383,603, filed Sep. 15, 2010, the entirety of each of which is herein incorporated by reference.

### FIELD OF THE INVENTION

The present invention provides compositions and methods for stabilization of fluorescent dyes. In particular, the present invention provides buffer systems comprising thiourea to protect against degradation of ozone-labile fluorescent dyes.

### BACKGROUND OF THE INVENTION

The cyanine family (e.g. Cy5) of fluorescent dyes are widely used in DNA microarray experiments, next generation nucleic acid sequencing, and a wide variety of other molecular biology, biochemical, biophysical, and cell biology applications. The large molar extinction coefficients and ease of enzymatic incorporation of cyanine dyes allows high sensitivity detection of low copy targets even when sample amounts are limited (Mujumdar et al. Bioconj Chem. 1993; 4:105-111., Liang et al. PNAS. 2005; 102:5814-5819., herein incorporated by reference in its entirety). However, a number of reports have been published documenting the instability of Cy5 and other dyes when exposed to elevated ozone levels in the environment (Fare et al. Anal Chem. 2003; 75:4672-4675., Branham et al. BMC Biotechnology. 2007; 7:8., herein incorporated by reference in their entireties). Ozone degradation of dyes can result in misinterpretation of experiments, for example, causing distortion of gene expression (Cy5/Cy3) ratios (Fare et al. Anal Chem. 2003; 75:4672-4675., Branham et al. BMC Biotechnology. 2007; 7:8., herein incorporated by reference in their entireties). In summer months, when environmental ozone levels increase, sample labeling (e.g. in microarray hybridization experiments) can suffer disproportionately from Cy5 (or other ozone-labile dyes) signal loss over time, impacting quality of data acquired. Compositions and methods for reduction in oxidative degradation of fluorescent dyes are needed in the field.

### SUMMARY OF THE INVENTION

In some embodiments, the present invention provides compositions and methods for stabilizing fluorescent dyes. For example, in some embodiments the invention provides methods comprising placing the fluorescent dye in buffer comprising one or more thioureas. In some embodiments, the fluorescent dye comprises an ozone-labile fluorescent dye. In some embodiments, the fluorescent dye comprises Cy5.

In some embodiments, the present invention provides a composition comprising: i) a buffer; ii) a fluorescent dye; and iii) one or more thioureas. In some embodiments, component iii) is thiourea. In some embodiments, the fluorescent dye comprises an ozone-labile fluorescent dye. In some embodiments, the fluorescent dye comprises Cy5. In some embodiments, the composition is formulated for use in molecular biology, biochemistry, biophysics, or cell biology applications. In some embodiments, the composition is formulated for use in DNA microarray analysis. In some embodiments, the composition is formulated for use in next generation nucleic acid sequencing. In some embodiments, the composition comprises one or more of ammonium persulfate, formamide, boric acid, glycine, citric acid, HEPES (2-[4-(2-hydroxyethyl)piperazin-1-yl]ethanesulfonic acid), Triton (e.g., Triton X-100; polyethylene glycol p-(1,1,3,3-tetramethylbutyl)-phenyl ether, octyl phenol ethoxylate, polyoxyethylene octyl phenyl ether, 4-octylphenol polyethoxylate,), SDS (sodium dodecyl sulfate), TWEEN (polyoxyethylene 20, 80, etc.), CHAPS (3[(3-Cholamidopropyl)dimethylammonio]-propanesulfonic acid), urea, MOPS (3-morpholinopropane-1-sulfonic acid), DTT (dithiothreitol; (2S,3S)-1,4-Bis-sulfanylbutane-2,3-diol), PIPES (1,4-Piperazinediethanesulfonic acid), EDTA, disodium salt, PBS Buffer (phosphate buffered saline), TEMED (N,N,N′,N′-Tetramethylethylenediamine), Tris HCl, sucrose, TBS Buffer (Tris, NaCl), TAE Buffer (Trizma, glacial acetic acid, EDTA), TBE Buffer (Tris, boric acid, EDTA), TG-SDS Buffer (Tris, glycine, SDS), phosphate buffer, magnesium chloride, magnesium sulfate, sodium chloride, sodium acetate, ammonium sulfate, and potassium chloride.

In some embodiments, the present invention provides a composition for use with fluorescent dyes comprising: i) a buffer and ii) one or more thioureas. In some embodiments, component ii) is thiourea. In some embodiments, the composition is formulated for use in molecular biology, biochemistry, biophysics, or cell biology applications. In some embodiments, the composition is formulated for use in DNA microarray analysis. In some embodiments, the composition is formulated for use in next generation nucleic acid sequencing. In some embodiments, the composition comprises one or more of ammonium persulfate, formamide, boric acid, glycine, citric acid, HEPES (2-[4-(2-hydroxyethyl)piperazin-1-yl]ethanesulfonic acid), Triton (e.g., Triton X-100; polyethylene glycol p-(1,1,3,3-tetramethylbutyl)-phenyl ether, octyl phenol ethoxylate, polyoxyethylene octyl phenyl ether, 4-octylphenol polyethoxylate,), SDS (sodium dodecyl sulfate), TWEEN (polyoxyethylene 20, 80, etc.), CHAPS (3[(3-Cholamidopropyl)dimethylammonio]-propanesulfonic acid), urea, MOPS (3-morpholinopropane-1-sulfonic acid), DTT (dithiothreitol; (2S,3S)-1,4-Bis-sulfanylbutane-2,3-diol), PIPES (1,4-Piperazinediethanesulfonic acid), EDTA, disodium salt, PBS Buffer (phosphate buffered saline), TEMED (N,N,N′,N′-Tetramethylethylenediamine), Tris HCl, sucrose, TBS Buffer (Tris, NaCl), TAE Buffer (Trizma, glacial acetic acid, EDTA), TBE Buffer (Tris, boric acid, EDTA), TG-SDS Buffer (Tris, glycine, SDS), phosphate buffer, magnesium chloride, magnesium sulfate, sodium chloride, sodium acetate, ammonium sulfate, and potassium chloride.

In some embodiments, the present invention provides a buffer for use with fluorescent dyes comprising one or more thioureas. In some embodiments, the buffer is formulated for use in molecular biology, biochemistry, biophysics, or cell biology applications. In some embodiments, the buffer is formulated for use in DNA microarray analysis. In some embodiments, the buffer is formulated for use in next generation nucleic acid sequencing. In some embodiments, the buffer comprises one or more of ammonium persulfate, formamide, boric acid, glycine, citric acid, HEPES (2-[4-(2-hydroxyethyl)piperazin-1-yl]ethanesulfonic acid), Triton (e.g., Triton X-100; polyethylene glycol p-(1,1,3,3-tetramethylbutyl)-phenyl ether, octyl phenol ethoxylate, polyoxyethylene octyl phenyl ether, 4-octylphenol polyethoxylate,), SDS (sodium dodecyl sulfate), TWEEN (polyoxyethylene 20, 80, etc.), CHAPS (3[(3-Cholamidopropyl)dimethylammonio]-propanesulfonic acid), urea, MOPS (3-morpholinopropane-1-sulfonic acid), DTT (dithiothreitol; (2S,3S)-1,4-Bis-sulfanylbutane-2,3-diol), PIPES (1,4-Piperazinediethanesulfonic acid), EDTA, disodium salt, PBS Buffer (phosphate buffered saline), TEMED (N,N,N′,N′-Tetramethylethylenediamine), Tris HCl, sucrose, TBS Buffer (Tris, NaCl), TAE Buffer (Trizma, glacial acetic acid, EDTA), TBE Buffer (Tris, boric acid, EDTA), TG-SDS Buffer (Tris, glycine, SDS), phosphate buffer, magnesium chloride, magnesium sulfate, sodium chloride, sodium acetate, ammonium sulfate, and potassium chloride.

In some embodiments, the present invention provides a kit comprising one or more fluorescent dyes and buffer comprising one or more thioureas. In some embodiments, at least one of the one or more fluorescent dyes comprises an ozone-labile fluorescent dye. In some embodiments, one of the one or more fluorescent dyes comprises Cy5. In some embodiments, the one or more thioureas comprises thiourea. In some embodiments, the buffer is formulated for use in molecular biology, biochemistry, biophysics, or cell biology applications. In some embodiments, the buffer is formulated for use in DNA microarray analysis. In some embodiments, the buffer is formulated for use in next generation nucleic acid sequencing. In some embodiments, the buffer comprises one or more of ammonium persulfate, formamide, boric acid, glycine, citric acid, HEPES (2-[4-(2-hydroxyethyl)piperazin-1-yl]ethanesulfonic acid), Triton (e.g., Triton X-100; polyethylene glycol p-(1,1,3,3-tetramethylbutyl)-phenyl ether, octyl phenol ethoxylate, polyoxyethylene octyl phenyl ether, 4-octylphenol polyethoxylate,), SDS (sodium dodecyl sulfate), TWEEN (polyoxyethylene 20, 80, etc.), CHAPS (3[(3-Cholamidopropyl)dimethylammonio]-propanesulfonic acid), urea, MOPS (3-morpholinopropane-1-sulfonic acid), DTT (dithiothreitol; (2S,35)-1,4-Bis-sulfanylbutane-2,3-diol), PIPES (1,4-Piperazinediethanesulfonic acid), EDTA, disodium salt, PBS Buffer (phosphate buffered saline), TEMED (N,N,N′,N′-Tetramethylethylenediamine), Tris HCl, sucrose, TBS Buffer (Tris, NaCl), TAE Buffer (Trizma, glacial acetic acid, EDTA), TBE Buffer (Tris, boric acid, EDTA), TG-SDS Buffer (Tris, glycine, SDS), phosphate buffer, magnesium chloride, magnesium sulfate, sodium chloride, sodium acetate, ammonium sulfate, and potassium chloride.

In some embodiments, the present invention provides kits comprising one or more of thiourea-containing buffer, non-thiourea-containing buffer, fluorescent dyes, and other reagents. In some embodiments, kits comprise reagents and buffers for fluorescent labeling (e.g. protein labeling, nucleic acid labeling, etc.). In some embodiments, kits comprise all of the components necessary and/or sufficient for labelling a sample, including all dyes, buffers (thiourea-containing buffer, non-thiourea-containing buffer, etc.), reagents, controls (e.g. control nucleic acids, control buffer, control protein, control cells, etc.), instructions, software, etc. In some embodiments, an end user of a kit supplies one or more standard reagents for use with a kit. In some embodiments, an end user of a kit supplies one or more reagents specific to their particular purposes, for use with a kit. In some embodiments, a kit comprises buffers only (e.g. one or more thiourea-containing buffers and/or one or more non-thiourea-containing buffers). In some embodiments, a kit comprises buffer (e.g. thiourea-containing buffer, non-thiourea-containing buffer) and fluorescent dye. In some embodiments, the buffer is a storage buffer.

### BRIEF DESCRIPTION OF THE DRAWINGS

FIG. 1 shows microarray analysis performed in the presence and absence of thiourea. Thiourea was present during elution of the fluorescently labeled DNA, hybridization of probe to the microarray, and post hybridization washing of the microarray. Microarray performed in the presence of thiourea exhibited higher total signal and more consistent signal across each spot.

FIG. 2 shows (a) thiourea reduction of a peroxide to diol, and (b) exemplary ozonolysis of cyclohexene to 1,6-hexanedialalkene by ozone and thiourea. The exemplary ozonolysis reaction is an example of a generic ozonolysis reaction of an alkene to a carbonyl.

### DEFINITIONS

As used herein, a “sample” refers to anything subjected to fluorescent labeling compositions and methods described herein. In some embodiments, the sample comprises or is suspected to comprise one or more nucleic acids, proteins, carbohydrates, lipids, and/or other biomolecules or non-biological molecules. Samples can include, for example, any compounds, polymers, macromolecules, nucleic acids, proteins, carbohydrates, lipids, cells, viruses, cell culture, growth media, tissue, whole organisms, groups of organisms, blood or blood components, saliva, urine, feces, nasal swabs, anorectal swabs, vaginal swabs, cervical swabs, medical samples, environmental samples, industrial samples, purified and/or isolated nucleic acid, purified and/or isolated nucleic acid, in vitro components (e.g. protein, nucleic acid, molecular biology reagents, etc.), and the like. In some embodiments, the samples are “mixture” samples, which comprise components from more than one subject or individual or source. In some embodiments, the methods provided herein comprise purifying the sample or purifying the component(s) from the sample.

As used herein, the term “isolated,” refers to a sample or portion of a sample that is identified and separated from at least one contaminant commonly associated with it. For example, when used in relation to a nucleic acid, as in “an isolated DNA” or “isolated polynucleotide” refers to a nucleic acid sequence that is identified and separated from at least one contaminant nucleic acid with which it is ordinarily associated in its natural source. Isolated nucleic acid is present in a form or setting that is different from that in which it is found in nature in contrast, non-isolated nucleic acids are nucleic acids such as DNA and RNA found in the state they exist in nature. As another example, an “isolated protein” or “isolated polypeptide” refers to a peptide sequence that is identified and separated from at least one peptide contaminant with which it is ordinarily associated in its natural source. Isolated protein is present in a form or setting that is different from that in which it is found in nature in contrast, non-isolated peptides are peptides such as found in the state they exist in nature.

As used herein, the term “purified” or “to purify” refers to the removal of components (e.g., contaminants) from a sample. For example, nucleic acids are purified by removal of contaminating proteins and other cellular components. Peptides are purified when they are removed from contaminating nucleic acid and cellular components. Both nucleic acids and proteins are purified when they are removed or extracted from a cell, thereby increasing their purity. Compounds are purified when they are separated from other contaminating compounds.

As used herein, the terms “fluorescent dye,” “fluorescent label,” and “fluorophore” are synonymous, and refer to molecules, portions of molecules, and/or functional groups which absorb energy of a specific wavelength and re-emit energy at a different specific wavelength.

As used herein, the term “thioureas” refers to a broad class of compounds with the general structure (R¹R²N)(R³R⁴N)C═S; wherein R₁ comprises any of hydrogen, alkyl, alkenyl, alkynyl, phenyl, benzyl, halide (e.g. fluoride, chloride, bromide, iodide), haloformyl, hydroxyl, carbonyl, aldehyde, carbonate ester, carboxylate, carboxyl, ether, ester, hydroperoxy, peroxy, carboxamide, amine, ketimine, aldimine, imide, azide, azo, cyanate, isocyanide, isocyanate, isothiocyante, nitrate, nitrile, nitrosooxy, nitro, nitroso, pyridyl, phosphino, phosphate, phosphono, sulfonyl, sulfo, sulfinyl, sulfhydryl, thiocyanate, disulfide, and combinations thereof; wherein R₂ comprises any of hydrogen, alkyl, alkenyl, alkynyl, phenyl, benzyl, halide (e.g. fluoride, chloride, bromide, iodide), haloformyl, hydroxyl, carbonyl, aldehyde, carbonate ester, carboxylate, carboxyl, ether, ester, hydroperoxy, peroxy, carboxamide, amine, ketimine, aldimine, imide, azide, azo, cyanate, isocyanide, isocyanate, isothiocyante, nitrate, nitrile, nitrosooxy, nitro, nitroso, pyridyl, phosphino, phosphate, phosphono, sulfonyl, sulfo, sulfinyl, sulfhydryl, thiocyanate, disulfide, and combinations thereof; wherein R₃ comprises any of hydrogen, alkyl, alkenyl, alkynyl, phenyl, benzyl, halide (e.g. fluoride, chloride, bromide, iodide), haloformyl, hydroxyl, carbonyl, aldehyde, carbonate ester, carboxylate, carboxyl, ether, ester, hydroperoxy, peroxy, carboxamide, amine, ketimine, aldimine, imide, azide, azo, cyanate, isocyanide, isocyanate, isothiocyante, nitrate, nitrile, nitrosooxy, nitro, nitroso, pyridyl, phosphino, phosphate, phosphono, sulfonyl, sulfo, sulfinyl, sulfhydryl, thiocyanate, disulfide, and combinations thereof; wherein R₄ comprises any of hydrogen, alkyl, alkenyl, alkynyl, phenyl, benzyl, halide (e.g. fluoride, chloride, bromide, iodide), haloformyl, hydroxyl, carbonyl, aldehyde, carbonate ester, carboxylate, carboxyl, ether, ester, hydroperoxy, peroxy, carboxamide, amine, ketimine, aldimine, imide, azide, azo, cyanate, isocyanide, isocyanate, isothiocyante, nitrate, nitrile, nitrosooxy, nitro, nitroso, pyridyl, phosphino, phosphate, phosphono, sulfonyl, sulfo, sulfinyl, sulfhydryl, thiocyanate, disulfide, and combinations thereof. Thiourea [(H₂N)₂C═S] is a common member of the class of thioureas. In some embodiments, thioureas are sulfathiourea, noxytiolin, or Burimamide.

As used herein, the term “ozone-labile fluorescent dye” refers to any fluorescent dye that undergoes degradation in the presence of ozone. The degradation may affect the fluorescence, structure, labeling capacity, and/or any other attribute or property of the fluorescent dye. Ozone-induced degradation of a fluorescent dye is typically evident from a reduction in the level of fluorescence of a labeled sample observed using traditional laboratory assays and detection systems, such as those described herein.

As used herein, the term “oxidation-susceptible fluorescent dye” refers to any fluorescent dye that undergoes some form of oxidative degradation. The degradation may affect the fluorescence, structure, labeling capacity, and/or any other attribute or property of the fluorescent dye. The degradation may be induced by a specific molecule (e.g. ozone, oxygen radical, etc.) or may occur more generally in the presence of molecules capable of initiating oxidation (e.g. in the air, in aqueous solution, etc.). Oxidative degradation of a fluorescent dye is typically evident from a reduction in the level of fluorescence of a labeled sample observed using traditional laboratory assays and detection systems, such as those described herein.

### DETAILED DESCRIPTION OF THE INVENTION

In some embodiments, the present invention provides compositions, methods, and kits for stabilization of ozone-labile fluorescent dyes. In some embodiments, the present invention prevents or reduces degradation of fluorescent dyes. In some embodiments, the present invention prevents or reduces oxidative degradation of fluorescent dyes. In some embodiments, the present invention prevents or reduces oxidative degradation of fluorescent dyes by oxygen radicals. In some embodiments, the present invention prevents or reduces oxidative degradation of fluorescent dyes by ozone. In some embodiments, the present invention stabilizes fluorescent dyes (e.g. in the presence of ozone and/or oxygen radicals). In some embodiments, the present invention provides buffers and buffer conditions that stabilize fluorescent dyes (e.g. in the presence of ozone and/or oxygen radicals). In some embodiments, the present invention provides buffer conditions to stabilize ozone-labile fluorescent dyes (e.g. Cy5).

In some embodiments, the present invention employs thiourea to control oxidative degradation of fluorescent dyes. In some embodiments, the present invention provides thiourea, thiourea-like compounds, molecules containing thiourea or thiourea-like substituent(s), derivatives of thiourea, etc. In some embodiments, thiourea-like compounds, molecules containing thiourea substituents, molecules containing thiourea-like substituents, and derivatives of thiourea may substitute for thiourea in embodiments described herein. In some embodiments, thioureas, molecules of Formula 1, find use in the present invention. Formula 1 comprises:

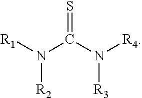

wherein R₁ comprises any of hydrogen, alkyl, alkenyl, alkynyl, phenyl, benzyl, halide (e.g. fluoride, chloride, bromide, iodide), haloformyl, hydroxyl, carbonyl, aldehyde, carbonate ester, carboxylate, carboxyl, ether, ester, hydroperoxy, peroxy, carboxamide, amine, ketimine, aldimine, imide, azide, azo, cyanate, isocyanide, isocyanate, isothiocyante, nitrate, nitrile, nitrosooxy, nitro, nitroso, pyridyl, phosphino, phosphate, phosphono, sulfonyl, sulfo, sulfinyl, sulfhydryl, thiocyanate, disulfide, and combinations thereof; wherein R₂ comprises any of hydrogen, alkyl, alkenyl, alkynyl, phenyl, benzyl, halide (e.g. fluoride, chloride, bromide, iodide), haloformyl, hydroxyl, carbonyl, aldehyde, carbonate ester, carboxylate, carboxyl, ether, ester, hydroperoxy, peroxy, carboxamide, amine, ketimine, aldimine, imide, azide, azo, cyanate, isocyanide, isocyanate, isothiocyante, nitrate, nitrile, nitrosooxy, nitro, nitroso, pyridyl, phosphino, phosphate, phosphono, sulfonyl, sulfo, sulfinyl, sulfhydryl, thiocyanate, disulfide, and combinations thereof; wherein R₃ comprises any of hydrogen, alkyl, alkenyl, alkynyl, phenyl, benzyl, halide (e.g. fluoride, chloride, bromide, iodide), haloformyl, hydroxyl, carbonyl, aldehyde, carbonate ester, carboxylate, carboxyl, ether, ester, hydroperoxy, peroxy, carboxamide, amine, ketimine, aldimine, imide, azide, azo, cyanate, isocyanide, isocyanate, isothiocyante, nitrate, nitrile, nitrosooxy, nitro, nitroso, pyridyl, phosphino, phosphate, phosphono, sulfonyl, sulfo, sulfinyl, sulfhydryl, thiocyanate, disulfide, and combinations thereof; wherein R₄ comprises any of hydrogen, alkyl, alkenyl, alkynyl, phenyl, benzyl, halide (e.g. fluoride, chloride, bromide, iodide), haloformyl, hydroxyl, carbonyl, aldehyde, carbonate ester, carboxylate, carboxyl, ether, ester, hydroperoxy, peroxy, carboxamide, amine, ketimine, aldimine, imide, azide, azo, cyanate, isocyanide, isocyanate, isothiocyante, nitrate, nitrile, nitrosooxy, nitro, nitroso, pyridyl, phosphino, phosphate, phosphono, sulfonyl, sulfo, sulfinyl, sulfhydryl, thiocyanate, disulfide, and combinations thereof. In some embodiments, thioureas such as sulfathiourea, noxytiolin, and Burimamide find use in embodiments of the present invention.

In some embodiments, thiourea is protective of fluorescent dyes (e.g. Cy5). In some embodiments, thiourea prevents or reduces oxidative degradation of fluorescent dyes. In some embodiments, thiourea prevents or reduces degradation of fluorescent dyes by ozone. In some embodiments, thiourea prevents or reduces degradation of fluorescent dyes by ozone-related molecules (e.g. molecules containing ozone-like substituents). In some embodiments, thiourea prevents or reduces degradation of fluorescent dyes by oxygen radicals, and molecules containing oxygen radical substituents. In some embodiments, thiourea is protective of ozone-labile fluorescent dyes (e.g. Cy5). In some embodiments, thiourea prevents or reduces oxidative degradation of oxidation-susceptible fluorescent dyes. In some embodiments, thiourea prevents or reduces degradation of ozone-labile fluorescent dyes by ozone. In some embodiments, thiourea prevents or reduces degradation of ozone-labile fluorescent dyes by ozone-related molecules (e.g. molecules containing ozone-like substituents). In some embodiments, thiourea prevents or reduces degradation of oxidation-susceptible fluorescent dyes by oxygen radicals, and molecules containing oxygen radical substituents. In some embodiments, thiourea prevents or reduces degradation of oxidation-susceptible fluorescent dyes by molecules generally capable of inducing oxidation (e.g. water).

In some embodiments, the present invention finds use with any fluorescent dyes, particularly fluorescent dyes which are susceptible to degradation in the presence of ozone, oxygen radicals, and/or oxidation-inducing molecules. However, compositions and methods of the present invention may also be used in the presence of dyes that are not susceptible to oxidative degradation. In some embodiments, the present invention finds use with acridine dyes, cyanine dyes, fluorone dyes, oxazin dyes, phenanthridine dyes, and/or rhodamine dyes. In some embodiments, the present invention finds use with: ATTO dyes, acridine orange, acridine yellow, Alexa Fluor, 7-aminoactinomycin D, 8-anilinonaphthalene-1-sulfonate, auramine-rhodamine stain, benzanthrone, 5,12-bis(phenylethynyl)naphthacene, 9,10-bis(phenylethynyl)anthracene, blacklight paint, brainbow, calcein, carboxyfluorescein, carboxyfluorescein diacetate succinimidyl ester, carboxyfluorescein succinimidyl ester, 1-chloro-9,10-bis(phenylethynyl)anthracene, 2-chloro-9,10-bis(phenylethynyl)anthracene, 2-chloro-9,10-diphenylanthracene, coumarin, Cy3, Cy5, DAPI, dark quencher, DiOC6, DyLight Fluor, Fluo-4, Fluoprobes, fluorescein, fluorescein isothiocyanate, Fluoro-Jade stain, Fura-2, Fura-2-acetoxymethyl ester, green fluorescent protein, Hoechst stain, Indian yellow, Indo-1, Luciferin, merocyanine, Nile blue, Nile red, optical brightener, perylene, phycobilin, phycoerythrin, phycoerythrobilin, pyranine, rhodamine, rhodamine 123, rhodamine 6G, RiboGreen, RoGFP, Rubrene, SYBR Green I, (E)-Stilbene, (Z)-Stilbene, sulforhodamine 101, sulforhodamine B, Synapto-pHluorin, tetraphenyl butadiene, tetrasodium tris(bathophenanthroline disulfonate)ruthenium(II), Texas Red, TSQ, umbelliferone, and/or yellow fluorescent protein.

In some embodiments, thiourea is provided as part of a buffer or buffer system comprising one or more additional components. In some embodiments, thiourea is provided in a buffer with components configured for molecular biology, biochemistry, biophysical and/or cell biology applications (e.g. sequencing, microarray, fluorescence resonance energy transfer (FRET), single molecule manipulations, etc.). In some embodiments, thiourea is provided as part of a buffer or buffer system comprising one or more of ammonium persulfate, formamide, boric acid, glycine, citric acid, HEPES (2-[4-(2-hydroxyethyl)piperazin-1-yl]ethanesulfonic acid), Triton (e.g., Triton X-100; polyethylene glycol p-(1,1,3,3-tetramethylbutyl)-phenyl ether, octyl phenol ethoxylate, polyoxyethylene octyl phenyl ether, 4-octylphenol polyethoxylate,), SDS (sodium dodecyl sulfate), TWEEN (polyoxyethylene 20, 80, etc.), CHAPS (3[(3-Cholamidopropyl)dimethylammonio]-propanesulfonic acid), urea, MOPS (3-morpholinopropane-1-sulfonic acid), DTT (dithiothreitol; (2S,3S)-1,4-Bis-sulfanylbutane-2,3-diol), PIPES (1,4-Piperazinediethanesulfonic acid), EDTA, disodium salt, PBS Buffer (phosphate buffered saline), TEMED (N,N,N′,N′-Tetramethylethylenediamine), Tris HCl, sucrose, TBS Buffer (Tris, NaCl), TAE Buffer (Trizma, glacial acetic acid, EDTA), TBE Buffer (Tris, boric acid, EDTA), TG-SDS Buffer (Tris, glycine, SDS), phosphate buffer, magnesium chloride, magnesium sulfate, sodium chloride, sodium acetate, ammonium sulfate, and potassium chloride, other common buffer components known to those of skill in the art, and buffer components configured for the specific application (e.g. sequencing, microarray, fluorescence resonance energy transfer (FRET), single molecule manipulations, etc.).

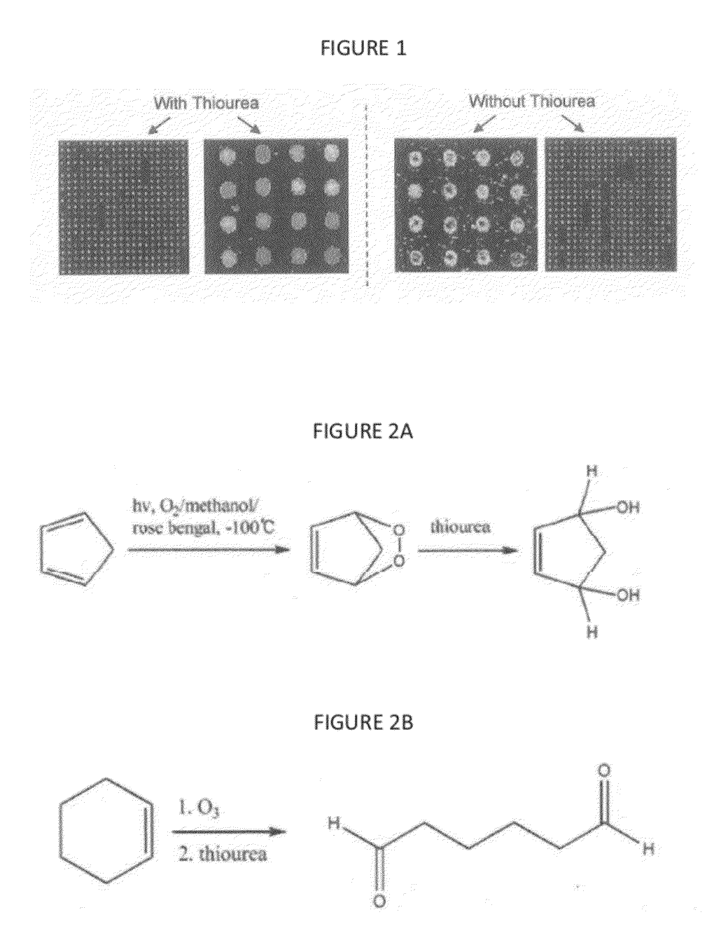

In some embodiments, inclusion of thiourea in molecular biology, biochemistry, biophysical and/or cell biology applications involving fluorophores (e.g. sequencing, microarray, fluorescence resonance energy transfer (FRET), single molecule manipulations, etc.) results in higher signal (e.g. higher total signal, higher signal of a specific fluorophore, etc.), increased consistency, prolonged effective experiment time, etc. In some embodiments, addition of thiourea during microarray analysis, resulted in higher total signal and more consistent signal across the array (SEE FIG. 1). In some embodiments, thiourea is included in buffer during preparation of reagents, dilution of fluorophores, reaction of components, labeling with fluorophores, washing of components, detection of fluorophores, analysis of results, and/or other steps in molecular biology, biochemistry, biophysical and/or cell biology applications. In some embodiments, thiourea is added during steps of a microarray analysis, for example, during elution of fluorescently labeled DNA, hybridization of probes to the microarray, post hybridization microarray washing, etc (SEE FIG. 1).

In some embodiments, thiourea is added to samples containing fluorescent labels in a concentration proportional to the amount of label. In some embodiments, thiourea is added to samples containing fluorescent labels in a concentration proportional to the amount of ozone present or presumed to be present. In some embodiments, thiourea is added to samples containing fluorescent labels in a concentration proportional to the amount of oxidation-inducing species present or presumed to be present. In some embodiments, a sufficient concentration of thiourea is added to samples containing fluorescent dyes to essentially eliminate oxidative degradation of fluorescent dyes. In some embodiments, a sufficient concentration of thiourea is added to samples containing fluorescent dyes to effectively reduce oxidative degradation of fluorescent dyes below the level of detection. In some embodiments, a sufficient concentration of thiourea is added to samples containing fluorescent dyes to effectively reduce oxidative degradation of fluorescent dyes below the level of other forms of fluorophore degradation. In some embodiments, thiourea is added to a sample at a concentration to reduce oxidative degradation (e.g. fluorophore degradation by ozone) by at least 10% (e.g. >10%, >20%, >50%, >75%, etc.) relative to the same sample in the absence of thiourea. In some embodiments, thiourea is added to a sample at a concentration to reduce oxidative degradation (e.g. fluorophore degradation by ozone) by at least 50% (e.g. >50%, >75%, >90%, >95%, >99%).

In some embodiments, thiourea is added to buffers and/or buffer systems along with one or more additional components to protect fluorophores from oxidative degradation (e.g. dimethyl sulfate). In some embodiments, thiourea is added to buffers and/or buffer systems along with one or more additional components to protect fluorophores from other types of degradation (e.g. UV-initiated degradation).

Although the present invention is not limited to any particular mechanism of action, and an understanding of the mechanism of action is not necessary to practice the present invention, it is contemplated that thiourea functions by reducing peroxides and/or ozonides that are the product of reactions of alkenes with ozone (SEE FIG. 2). Ozone reacts with alkenes to produce an unstable ozonide intermediate. Thiourea reacts with the ozonide to form a carbonyl (SEE FIG. 2B). By converting ozonides to carbonyls, thiourea uses up the available ozone, thereby reducing the potential for ozone to interact with any fluorescent dye. By converting the unstable intermediate ozonide to a stable carbonyl, thiourea drives the reaction toward completion, and uses up available ozone, thereby minimizing the amount of ozone available for reacting with fluorescent dyes. Thus, in some embodiments, any agent having these capabilities may be used in addition to or in place of thiourea.

The compositions, methods, and kits of the present invention find use in molecular biology, biochemistry, biophysics, and cell biology applications, but are not limited to any particular fields. In some embodiments, compositions, methods, and kits of the present invention find use in medical, environmental, agricultural, law enforcement, and other fields. In some embodiments, the present invention finds use in diagnostics, research, clinical, and field applications. The compositions, methods, and kits of the present invention are not limited to any particular use.

For example, the compositions, methods, and kits may be applied to nucleic acid sequencing technologies. Illustrative non-limiting examples of nucleic acid sequencing techniques include, but are not limited to, chain terminator (Sanger) sequencing and dye terminator sequencing, as well as “next-generation sequencing” techniques. A set of methods referred to as “next-generation sequencing” techniques have emerged as alternatives to Sanger and dye-terminator sequencing methods (Voelkerding et al., Clinical Chem., 55: 641-658, 2009; MacLean et al., Nature Rev. Microbiol., 7: 287-296; each herein incorporated by reference in their entirety). Most current methods describe the use of next-generation sequencing technology for de novo sequencing of whole genomes to determine the primary nucleic acid sequence of an organism. In addition, targeted re-sequencing (deep sequencing) allows for sensitive mutation detection within a population of wild-type sequence. Some examples include recent work describing the identification of HIV drug-resistant variants as well as EGFR mutations for determining response to anti-TK therapeutic drugs. Recent publications describing the use of bar code primer sequences permit the simultaneous sequencing of multiple samples during a typical sequencing run including, for example: Margulies, M. et al. “Genome Sequencing in Microfabricated High-Density Picolitre Reactors”, Nature, 437, 376-80 (2005); Mikkelsen, T. et al. “Genome-Wide Maps of Chromatin State in Pluripotent and Lineage-Committed Cells”, Nature, 448, 553-60 (2007); McLaughlin, S. et al. “Whole-Genome Resequencing with Short Reads: Accurate Mutation Discovery with Mate Pairs and Quality Values”, ASHG Annual Meeting (2007); Shendure J. et al. “Accurate Multiplex Polony Sequencing of an Evolved Bacterial Genome”, Science, 309, 1728-32 (2005); Harris, T. et al. “Single-Molecule DNA Sequencing of a Viral Genome”, Science, 320, 106-9 (2008); Simen, B. et al. “Prevalence of Low Abundance Drug Resistant Variants by Ultra Deep Sequencing in Chronically HIV-infected Antiretroviral (ARV) Naïve Patients and the Impact on Virologic Outcomes”, 16th International HIV Drug Resistance Workshop, Barbados (2007); Thomas, R. et al. “Sensitive Mutation Detection in Heterogeneous Cancer Specimens by Massively Parallel Picoliter Reactor Sequencing”, Nature Med., 12, 852-855 (2006); Mitsuya, Y. et al. “Minority Human Immunodeficiency Virus Type 1 Variants in Antiretroviral-Naïve Persons with Reverse Transcriptase Codon 215 Revertant Mutations”, J. Vir., 82, 10747-10755 (2008); Binladen, J. et al. “The Use of Coded PCR Primers Enables High-Throughput Sequencing of Multiple Homolog Amplification Products by 454 Parallel Sequencing”, PLoS ONE, 2, e197 (2007); and Hoffmann, C. et al. “DNA Bar Coding and Pyrosequencing to Identify Rare HIV Drug Resistance Mutations”, Nuc. Acids Res., 35, e91 (2007), all of which are herein incorporated by reference.

Compared to traditional Sanger sequencing, next-gen sequencing technology produces large amounts of sequencing data points. A typical run can easily generate tens to hundreds of megabases per run, with a potential daily output reaching into the gigabase range. This translates to several orders of magnitude greater than a standard 96-well plate, which can generate several hundred data points in a typical multiplex run. Target amplicons that differ by as little as one nucleotide can easily be distinguished, even when multiple targets from related species are present. This greatly enhances the ability to do accurate genotyping. Next-gen sequence alignment software programs used to produce consensus sequences can easily identify novel point mutations, which could result in new strains with associated drug resistance. The use of primer bar coding also allows multiplexing of different patient samples within a single sequencing run.

Next-generation sequencing (NGS) methods share the common feature of massively parallel, high-throughput strategies, with the goal of lower costs in comparison to older sequencing methods. NGS methods can be broadly divided into those that require template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and emerging platforms commercialized by VisiGen and Pacific Biosciences, respectively.

In pyrosequencing (Voelkerding et al., Clinical Chem., 55: 641-658, 2009; MacLean et al., Nature Rev. Microbiol., 7: 287-296; U.S. Pat. No. 6,210,891; U.S. Pat. No. 6,258,568; each herein incorporated by reference in its entirety), template DNA is fragmented, end-repaired, ligated to adaptors, and clonally amplified in-situ by capturing single template molecules with beads bearing oligonucleotides complementary to the adaptors. Each bead bearing a single template type is compartmentalized into a water-in-oil microvesicle, and the template is clonally amplified using a technique referred to as emulsion PCR. The emulsion is disrupted after amplification and beads are deposited into individual wells of a picotitre plate functioning as a flow cell during the sequencing reactions. Ordered, iterative introduction of each of the four dNTP reagents occurs in the flow cell in the presence of sequencing enzymes and reporter molecules. In the event that an appropriate dNTP is added to the 3′ end of the sequencing primer, the resulting production of ATP causes a signal within the well, which is detected. It is possible to achieve read lengths greater than or equal to 400 bases, and 1×10⁶ sequence reads can be achieved, resulting in up to 500 million base pairs (Mb) of sequence.

In the Solexa/Illumina platform (Voelkerding et al., Clinical Chem., 55: 641-658, 2009; MacLean et al., Nature Rev. Microbiol., 7: 287-296; U.S. Pat. No. 6,833,246; U.S. Pat. No. 7,115,400; U.S. Pat. No. 6,969,488; each herein incorporated by reference in its entirety), sequencing data are produced in the form of shorter-length reads. In this method, single-stranded fragmented DNA is end-repaired to generate 5′-phosphorylated blunt ends, followed by Klenow-mediated addition of a single A base to the 3′ end of the fragments. A-addition facilitates addition of T-overhang adaptor oligonucleotides, which are subsequently used to capture the template-adaptor molecules on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluor and block removed prior to the next cycle of dNTP addition. Sequence read length ranges from 36 nucleotides to over 50 nucleotides, with overall output exceeding 1 billion nucleotide pairs per analytical run.

Sequencing nucleic acid molecules using SOLiD technology (Voelkerding et al., Clinical Chem., 55: 641-658, 2009; MacLean et al., Nature Rev. Microbiol., 7: 287-296; U.S. Pat. No. 5,912,148; U.S. Pat. No. 6,130,073; each herein incorporated by reference in their entirety) also involves fragmentation of the template, ligation to oligonucleotide adaptors, attachment to beads, and clonal amplification by emulsion PCR. Following this, beads bearing template are immobilized on a derivatized surface of a glass flow-cell, and a primer complementary to the adaptor oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLiD system, interrogation probes have 16 possible combinations of the two bases at the 3′ end of each probe, and one of four fluors at the 5′ end. Fluor color and thus identity of each probe corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, nanopore sequencing in employed (see, e.g., Astier et al., J Am Chem Soc. 2006 Feb. 8; 128(5):1705-10, herein incorporated by reference). The theory behind nanopore sequencing has to do with what occurs when the nanopore is immersed in a conducting fluid and a potential (voltage) is applied across it: under these conditions a slight electric current due to conduction of ions through the nanopore can be observed, and the amount of current is exceedingly sensitive to the size of the nanopore. If DNA molecules pass (or part of the DNA molecule passes) through the nanopore, this can create a change in the magnitude of the current through the nanopore, thereby allowing the sequences of the DNA molecule to be determined. The nanopore may be a solid-state pore fabricated on a metal and/or nonmetal surface, or a protein-based nanopore, such as α-hemolysin (Clarke et al., Nat. Nanotech., 4, Feb. 22, 2009: 265-270).

HeliScope by Helicos BioSciences (Voelkerding et al., Clinical Chem., 55: 641-658, 2009; MacLean et al., Nature Rev. Microbiol., 7: 287-296; U.S. Pat. No. 7,169,560; U.S. Pat. No. 7,282,337; U.S. Pat. No. 7,482,120; U.S. Pat. No. 7,501,245; U.S. Pat. No. 6,818,395; U.S. Pat. No. 6,911,345; U.S. Pat. No. 7,501,245; each herein incorporated by reference in their entirety) is the first commercialized single-molecule sequencing platform. This method does not require clonal amplification. Template DNA is fragmented and polyadenylated at the 3′ end, with the final adenosine bearing a fluorescent label. Denatured polyadenylated template fragments are ligated to poly(dT) oligonucleotides on the surface of a flow cell. Initial physical locations of captured template molecules are recorded by a CCD camera, and then label is cleaved and washed away. Sequencing is achieved by addition of polymerase and serial addition of fluorescently-labeled dNTP reagents. Incorporation events result in fluor signal corresponding to the dNTP, and signal is captured by a CCD camera before each round of dNTP addition. Sequence read length ranges from 25-50 nucleotides, with overall output exceeding 1 billion nucleotide pairs per analytical run. Other emerging single molecule sequencing methods real-time sequencing by synthesis using a VisiGen platform (Voelkerding et al., Clinical Chem., 55: 641-658, 2009; U.S. Pat. No. 7,329,492; U.S. patent application Ser. No. 11/671956; U.S. patent application Ser. No. 11/781,166; each herein incorporated by reference in their entirety) in which immobilized, primed DNA template is subjected to strand extension using a fluorescently-modified polymerase and fluorescent acceptor molecules, resulting in detectable fluorescence resonance energy transfer (FRET) upon nucleotide addition. Another real-time single molecule sequencing system developed by Pacific Biosciences (Voelkerding et al., Clinical Chem., 55: 641-658, 2009; MacLean et al., Nature Rev. Microbiol., 7: 287-296; U.S. Pat. No. 7,170,050; U.S. Pat. No. 7,302,146; U.S. Pat. No. 7,313,308; U.S. Pat. No. 7,476,503; all of which are herein incorporated by reference) utilizes reaction wells 50-100 nm in diameter and encompassing a reaction volume of approximately 20 zeptoliters (10×10²¹ L). Sequencing reactions are performed using immobilized template, modified phi29 DNA polymerase, and high local concentrations of fluorescently labeled dNTPs. High local concentrations and continuous reaction conditions allow incorporation events to be captured in real time by fluor signal detection using laser excitation, an optical waveguide, and a CCD camera.

In certain embodiments, the single molecule real time (SMRT) DNA sequencing methods using zero-mode waveguides (ZMWs) developed by Pacific Biosciences, or similar methods, are employed. With this technology, DNA sequencing is performed on SMRT chips, each containing thousands of zero-mode waveguides (ZMWs). A ZMW is a hole, tens of nanometers in diameter, fabricated in a 100 nm metal film deposited on a silicon dioxide substrate. Each ZMW becomes a nanophotonic visualization chamber providing a detection volume of just 20 zeptoliters (10-21 liters). At this volume, the activity of a single molecule can be detected amongst a background of thousands of labeled nucleotides.

The ZMW provides a window for watching DNA polymerase as it performs sequencing by synthesis. Within each chamber, a single DNA polymerase molecule is attached to the bottom surface such that it permanently resides within the detection volume. Phospholinked nucleotides, each type labeled with a different colored fluorophore, are then introduced into the reaction solution at high concentrations which promote enzyme speed, accuracy, and processivity. Due to the small size of the ZMW, even at these high, biologically relevant concentrations, the detection volume is occupied by nucleotides only a small fraction of the time. In addition, visits to the detection volume are fast, lasting only a few microseconds, due to the very small distance that diffusion has to carry the nucleotides. The result is a very low background.

As the DNA polymerase incorporates complementary nucleotides, each base is held within the detection volume for tens of milliseconds, which is orders of magnitude longer than the amount of time it takes a nucleotide to diffuse in and out of the detection volume. During this time, the engaged fluorophore emits fluorescent light whose color corresponds to the base identity. Then, as part of the natural incorporation cycle, the polymerase cleaves the bond holding the fluorophore in place and the dye diffuses out of the detection volume. Following incorporation, the signal immediately returns to baseline and the process repeats.

Unhampered and uninterrupted, the DNA polymerase continues incorporating bases at a speed of tens per second. In this way, a completely natural long chain of DNA is produced in minutes. Simultaneous and continuous detection occurs across all of the thousands of ZMWs on the SMRT chip in real time. Researchers at PacBio have demonstrated this approach has the capability to produce reads thousands of nucleotides in length.

Fluorescent dyes are also commonly used in probe-based nucleic acid detection technologies, including, but not limited to in situ methods (e.g., FISH), microarrays, and methods employing detection during or following nucleic acid amplification (e.g., polymerase chain reaction (PCR), reverse-transcriptase PCR (RT-PCR), transcription-based amplification (TAS), strand displacement amplification (SDA), ligase chain reaction (LCR), and the like. Probes may include one or more labels. Probes may be used singularly or in combinations. Probes may include secondary structure (e.g., molecule beacons) or be altered (digest, cleaved) to influence detection.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A composition comprising: i) a buffer; ii) a fluorescent dye; and iii) or more thioureas.

2. The composition of claim 1, wherein one of said one or more thioureas comprises thiourea.

3. The composition of claim 1, wherein said fluorescent dye comprises an ozone-labile fluorescent dye.

4. The composition of claim 3, wherein said fluorescent dye comprises Cy5.

5. The composition of claim 1, wherein said composition is formulated for use in molecular biology, biochemistry, biophysics, or cell biology applications.

6. The composition of claim 5, wherein said composition is formulated for use in DNA microarray analysis.

7. The composition of claim 5, wherein said composition is formulated for use in next generation nucleic acid sequencing.

8. The composition of claim 1, wherein said composition comprises one or more of ammonium persulfate, formamide, boric acid, glycine, citric acid, HEPES (2-[4-(2-hydroxyethyl)piperazin-1-yl]ethanesulfonic acid), Triton (e.g., Triton X-100; polyethylene glycol p-(1,1,3,3-tetramethylbutyl)-phenyl ether, octyl phenol ethoxylate, polyoxyethylene octyl phenyl ether, 4-octylphenol polyethoxylate,), SDS (sodium dodecyl sulfate), TWEEN (polyoxyethylene 20, 80, etc.), CHAPS (3[(3-Cholamidopropyl)dimethylammonio]-propanesulfonic acid), urea, MOPS (3-morpholinopropane-1-sulfonic acid), DTT (dithiothreitol; (2S,3S)-1,4-Bis-sulfanylbutane-2,3-diol), PIPES (1,4-Piperazinediethanesulfonic acid), EDTA, disodium salt, PBS Buffer (phosphate buffered saline), TEMED (N,N,N′,N′-Tetramethylethylenediamine), Tris HCl, sucrose, TBS Buffer (Tris, NaCl), TAE Buffer (Trizma, glacial acetic acid, EDTA), TBE Buffer (Tris, boric acid, EDTA), TG-SDS Buffer (Tris, glycine, SDS), phosphate buffer, magnesium chloride, magnesium sulfate, sodium chloride, sodium acetate, ammonium sulfate, and potassium chloride.

9. A composition for use with fluorescent dyes comprising: i) a buffer and ii) one or more thioureas.

10. The composition of claim 9, wherein one of said one or more thioureas comprises thiourea.

11. The composition of claim 9, wherein said composition is formulated for use in molecular biology, biochemistry, biophysics, or cell biology applications.

12. The composition of claim 11, wherein said composition is formulated for use in DNA microarray analysis.

13. The composition of claim 11, wherein said composition is formulated for use in next generation nucleic acid sequencing.

14. The composition of claim 9, wherein said composition comprises one or more of ammonium persulfate, formamide, boric acid, glycine, citric acid, HEPES (2-[4-(2-hydroxyethyl)piperazin-1-yl]ethanesulfonic acid), Triton (e.g., Triton X-100; polyethylene glycol p-(1,1,3,3-tetramethylbutyl)-phenyl ether, octyl phenol ethoxylate, polyoxyethylene octyl phenyl ether, 4-octylphenol polyethoxylate,), SDS (sodium dodecyl sulfate), TWEEN (polyoxyethylene 20, 80, etc.), CHAPS (3[(3-Cholamidopropyl)dimethylammonio]-propanesulfonic acid), urea, MOPS (3-morpholinopropane-1-sulfonic acid), DTT (dithiothreitol; (2S3S)-1,4-Bis-sulfanylbutane-2,3-diol), PIPES (1,4-Piperazinediethanesulfonic acid), EDTA, disodium salt, PBS Buffer (phosphate buffered saline), TEMED (N,N,N′,N′-Tetramethylethylenediamine), Tris HCl, sucrose, TBS Buffer (Tris, NaCl), TAE Buffer (Trizma, glacial acetic acid, EDTA), TBE Buffer (Tris, boric acid, EDTA), TG-SDS Buffer (Tris, glycine, SDS), phosphate buffer, magnesium chloride, magnesium sulfate, sodium chloride, sodium acetate, ammonium sulfate, and potassium chloride.

15. A method for stabilizing fluorescent dyes comprising placing said fluorescent dye in buffer comprising one or more thioureas.

16. The method of claim 15, wherein said fluorescent dye comprises an ozone-labile fluorescent dye.

17. The method of claim 16, wherein said fluorescent dye comprises Cy5.

18. The method of claim 15, wherein one of said one or more thioureas comprises thiourea.

19. A kit comprising one or more fluorescent dyes and buffer comprising one or more thioureas.

20. The kit of claim 19, wherein at least one of said one or more fluorescent dyes comprises an ozone-labile fluorescent dye.

21. The kit of claim 20, wherein one of said one or more fluorescent dyes comprises Cy5.

22. The kit of claim 19, wherein one of said one or more thioureas comprises thiourea.

23. The kit of claim 20, wherein said buffer is formulated for use in molecular biology, biochemistry, biophysics, or cell biology applications.

24. The kit of claim 23, wherein said buffer is formulated for use in DNA microarray analysis.

25. The kit of claim 23, wherein said buffer is formulated for use in next generation nucleic acid sequencing.

26. The kit of claim 19, comprising one or more of ammonium persulfate, formamide, boric acid, glycine, citric acid, HEPES (2-[4-(2-hydroxyethyl)piperazin-1-yl]ethanesulfonic acid), Triton (e.g., Triton X-100; polyethylene glycol p-(1,1,3,3-tetramethylbutyl)-phenyl ether, octyl phenol ethoxylate, polyoxyethylene octyl phenyl ether, 4-octylphenol polyethoxylate,), SDS (sodium dodecyl sulfate), TWEEN (polyoxyethylene 20, 80, etc.), CHAPS (3[(3-Cholamidopropyl)dimethylammonio]-propanesulfonic acid), urea, MOPS (3-morpholinopropane-1-sulfonic acid), DTT (dithiothreitol; (2S,3S)-1,4-Bis-sulfanylbutane-2,3-diol), PIPES (1,4-Piperazinediethanesulfonic acid), EDTA, disodium salt, PBS Buffer (phosphate buffered saline), TEMED (N,N,N′,N′-Tetramethylethylenediamine), Tris HCl, sucrose, TBS Buffer (Tris, NaCl), TAE Buffer (Trizma, glacial acetic acid, EDTA), TBE Buffer (Tris, boric acid, EDTA), TG-SDS Buffer (Tris, glycine, SDS), phosphate buffer, magnesium chloride, magnesium sulfate, sodium chloride, sodium acetate, ammonium sulfate, and potassium chloride.
